(19) United States
(12) Patent Application Publication
(10) Pub. No.: US 2004/0067158 A1
(43) Pub. Date: Apr. 8, 2004

(54) Sterilizer for medical appliance

(76) Inventor: Yong-ik Yi, Seoul (KR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/433,410

(22) PCT Filed: Jul. 3, 2001
(86) PCT No.: PCT/KR01/01132

(30) Foreign Application Priority Data
Dec. 4, 2000 (KR) 2000/72836

(51) Int. Cl. A61L 2/07

(52) U.S. Cl. 422/26; 422/298

(57) ABSTRACT

The present invention relates to a sterilizer for medical appliances. More specifically, the present invention relates to a sterilizer for medical appliances to reduce noise and perform sterilization in a short time. A sterilizer for medical appliances, comprising a water reservoir which stores distilled water for sterilization; a chamber for heating the distilled water fed from said water reservoir for sterilization of medical appliances to produce vapor; a cooler for reducing the high temperature of the vapor produced in said chamber, and a vacuum pump for reducing pressure and moisture in said chamber, wherein the sterilizer comprises a vapor-liquid separator for separating liquid from wet vapor produced in said chamber; and muffler for reducing noises generated at the discharge side of said vacuum pump.

This invention relates to a sterilizer for medical appliances, and in particular to a sterilizer for medical appliances which may minimize noise that occurs during sterilization, and also perform sterilization in a short of time.

### FIELD OF TEE INVASION

Generally, various appliances for medical purposes (referred to as medical appliances hereafter) must be stored and used after sterilization. The common method for sterilizing medical appliances comprises the method of using gas, the method of using chemicals, and the method of using high temperature steams. During sterilization of medical appliances, although the gas method is used to sterilize medical appliances that are vulnerable to high temperature and moisture and thus making the steam sterilization method inapplicable, sterilization using high temperature steam is preferable for most medical appliances used on the human body.

The conventional sterilizer, as depicted in FIG. 1, includes a chamber (2) having a heater formed to sterilize medical appliances received therein, a water reservoir (8) which stores distilled water that is supplied to produce wet steam within said chamber (2), a vacuum pump (6) formed to lower pressure and moisture within said chamber (2), a cooler (10) installed to lower the temperature of the wet steam when recovering the wet steam from said chamber (2), and solenoid valves (4,12) formed to respectively supply and recover wet steam and distilled water.

In the conventional sterilizer as the above, because wet steam is directly conveyed to the vacuum pump (6) in the process of discharging the wet steam generated in the chamber (2) during sterilization, there is the problem of decreasing the efficiency of the vacuum pump (6). That is, although the temperature of the wet steam may be lowered and a portion of the wet steam may be made into liquid by means of the cooler (10) installed between the vacuum pump (6) and chamber (2), all of the wet steam may not be removed by the cooler (10) and thus the efficiency of the vacuum pump (6) is decreased. In addition, when the vacuum pump (6) is operated to make the chamber (2) into a vacuum state, noise is occured. The level of the noise occured amounts to approximately 75 dB, and the noise increases as the moisture of the wet steam entering the vacuum pump (6) increases. Conventionally, this noise is unable to be eliminated and thus there is the problem of causing discomfort and stress to the operators.

In addition, because moisture remains on the medical appliances after sterilization by the wet steam in the chamber (2), the medical appliances may get infected by viruses. Therefore, to prevent virus infection, the medical appliances must be dried and thus there is the problem of the sterilization time becoming longer.

### SUMMARY OF THE INVENION

The present invention has been contrived to resolve the above problems, and the object thereof is to improve sterilization ability by preventing medical appliances from being infected by viruses during sterilization of medical appliances.

In addition, another object of the present invention is to provide a sterilizer for medical appliances which reduces sterilization time and decreases noise occured from the sterilizer.

To achieve the above objects, a sterilize for medical appliances according to the present invention comprises a water reservoir which stores distilled water for sterilization, a chamber for heating the distilled water fed from said water reservoir for to sterilization of medical appliances, to produce vapor a cooler for reducing the temperature of the hot vapor steam produced in said chamber, and a vacuum pump for reducing the pressure and moisture in said chamber, said sterilizer for medical appliances further comprising a vapor-liquid separator which is arranged between the vacuum pump and the cooler and separates liquid from wet vapor produced in said chamber and a muffler for reducing noises generated at the discharge side of said vacuum pump.

### BRIEF DESCRIPTION OF THE DRAWINGS

FIG. 1 is a drawing showing the conventional sterilizer for medical appliance;

FIG. 2 is a drawing showing the sterilizer for medical appliance according to the present invention;

FIG. 3 shows the structure of the sterilizer for medical appliance according to the present invention;

FIG. 4 is an enlarged view of the vapor-liquid separator of the sterilizer for medical appliance according to the present invention;

FIG. 5 is an enlarged view of the muffler of the medical sterilizer for medical appliance according to the present invention.

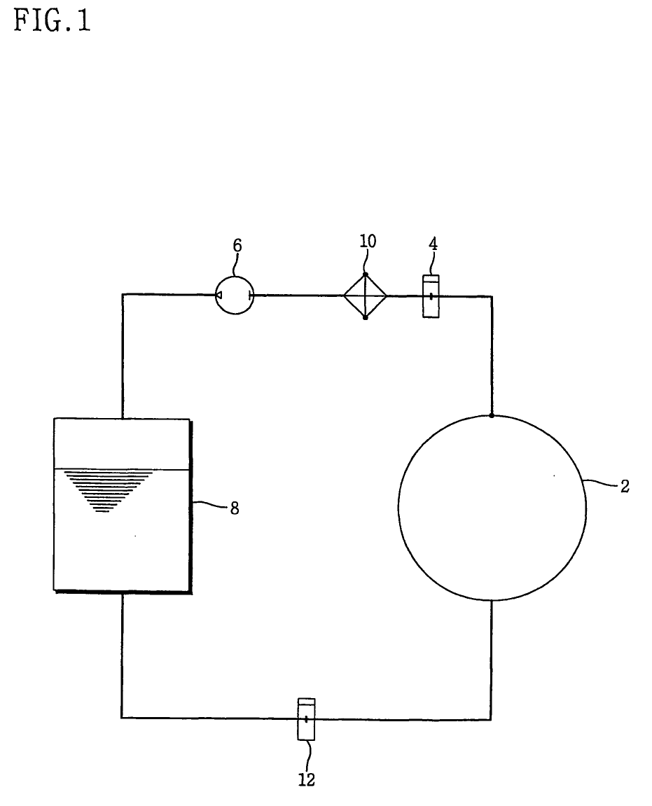

<Description of the reference numbers>

| | |
|---|---|
| 30: water reservoir | 36: chamber |
| 40: cooler | 42: vapor-liquid separator |
| 44: vacuum pump | 46: muffler |

### DETAILED DESCRIPTION OF THE PREFERRED EMBODIMENTS

The sterilizer for medical appliance according to the present invention designated as the above is described hereinafter with reference to the attached drawings.

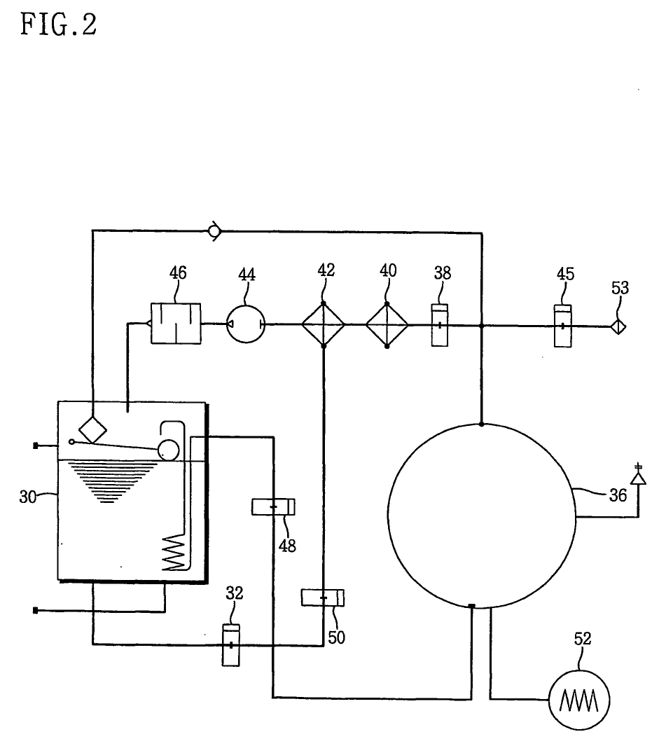

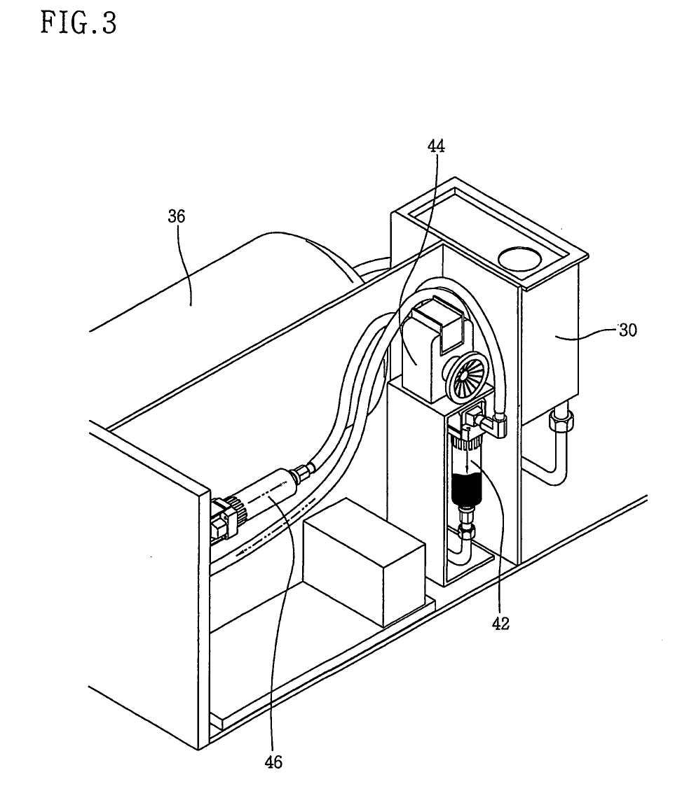

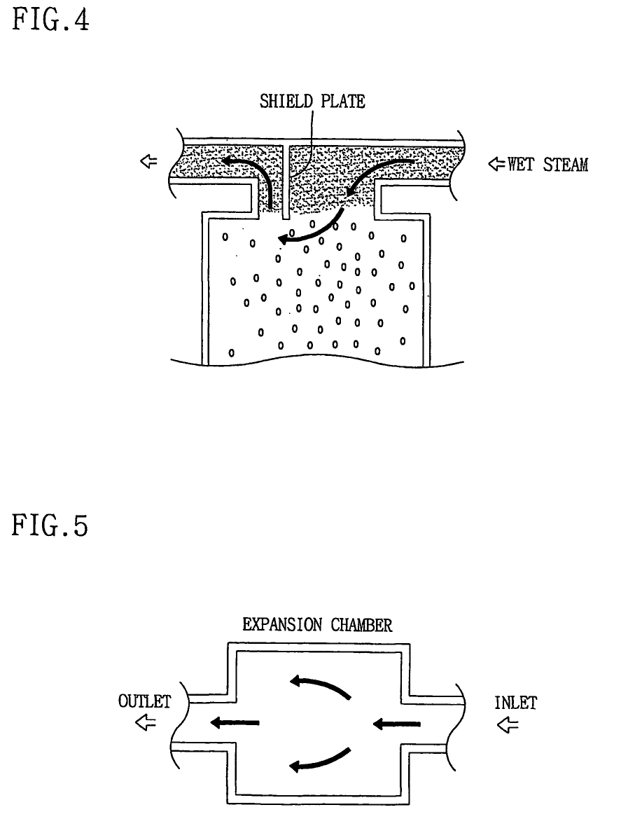

FIG. 2 is a drawing showing the sterilizer for medical appliance according to the present invention, FIG. 3 is a drawing showing the structure of the sterilizer for medical appliance according to the present invention, FIG. 4 is an enlarged view of the vapor-liquid separator of the sterilizer for medical appliance according to the present invention, and FIG. 5 is an enlarged view of the muffler of the sterilize for medical appliance according to the present invention.

The sterilizer of the present invention employs the heating method, wherein the distilled water stored in the water reservoir (30) is fed to the chamber (36) which heats the distilled water for sterilization of medical appliances. A solenoid valve (32) is arranged between said water reservoir (30) which stores the distilled water and the chamber (36) for sterilizing medical appliances received therein, and controls the amount of distilled water which becomes wet vapor from heating within the chamber (36). The distilled water fed from the water reservoir (30) is heated in the chamber (36) and transformed into hot wet vapor to sterilize medical appliances.

The operation of the sterilizer for medical appliance according to the present invention is described hereinafter. Firstly, before the distilled water within the water reservoir (30) is moved to the chamber (36), the vacuum pump (44) is activated to make the pressure in the chamber (36) lower than atmospheric pressure to a vacuumed state of approximately 480 mmHg. Then, when the pressure in the chamber (36) is lowered by said vacuum pump (44), the temperature within

the chamber (36) is controlled to approximately 121° C. to approximately 134° C., and pressure is controlled to approximately 1 kg(f)/cm² to approximately 2 kg(f)m², such that a proper state for sterilization is made.

A thermometer (52) is attached to the chamber (36), and the of the chamber (36) is controlled by a steam trap (not shown) to maintain a set predetermined temperature and prevent sudden changes in the temperature of the chamber (36).

Then, the wet steam generated in the chamber (36) is discharged by the vacuum pump (44). In the process of discharging the wet steam generated in the chamber (36), to prevent the wet steam from directly entering the vacuum pump (44), the wet steam is cooled by the cooler (40). Then, the dry steam separated by the vapor-liquid separated (42) enters the vacuum pump (44). The wet steam cooled by the cooler (40) collides with the internal walls of the vapor-liquid separator (42) and turns into dewdrops as it passes through the vapor-liquid separator (42) and thus the water is stored in the bottom portion of the vapor-liquid separator (42). Detailed description of the above is described hereinafter with reference to the attached drawings. The flow velocity of the wet steam moved via the cooler (40) is largely deceased as it enters inside of the vapor-liquid separator (42) which has a lager sectional area Man the tube, and the wet steam of reduced flow velocity collides with a shield plate and loses its own energy and turns into dewdrops and is finally stored in the bottom portion.

Therefore, because the wet steam can be separated by the vapor-liquid separator (42), the moisture of the wet steam generated within the chamber (36) is not entered into the vacuum pump (44). As a result, the vacuum pump (44) is not overworked and thus the chamber (36) may be smoothly vacuumed.

A solenoid valve (50) is installed in the lower discharge passage of said vapor-liquid separator (42), and the solenoid valve (50) feeds the moisture separated by the vapor-liquid separator (42) to the chamber (36).

The muffler (46) discharges noises generated at the discharge side of the vacuum pump (44) trough a tube passage. The noise generated at the discharge side of the vacuum pump expands in volume as it enters the muffler (46), and the noise is decreased (refer to FIG. 5). As a result, only the decreased noise and condensed moisture are discharged through the discharge side and are collected to the water reservoir (30). Therefore, the noise generated at the discharge side of the vacuum pump (44) and the condensed water which passes through the vacuum pump (44) are reduced in noise by the muffler (46) and collected into the water reservoir (30). Here, the noise is reduced from 75 dB, prior to installing the muffler, to 42 dB.

In the above process during sterilization of medical appliances within said chamber (36), the solenoid valve (48) is opened to allow the distilled water fed into the chamber (36) to be stored in the water reservoir (30), and after the distilled water is collected, sterilization is performed repeatedly.

After extracting the distilled water from within the chamber (36) as the above, the vacuum pump (44) is used to vacuum the chamber (36) and, in this vacuum state, the chamber is subjected to a drying process to remove the moisture remaining on the medical appliances received therein. In addition, when drying the chamber (36), to improve drying efficiency, a solenoid valve (45) and a bacterial filter (53) are installed to change the vacuum state to atmospheric pressure. Therefore, when the solenoid valve (38) is closed in the vacuum state, and the solenoid valve (45) is opened to increase dryness by changing the atmospheric pressure, because the chamber (36) becomes the same as the atmospheric pressure and the atmospheric pressure which is high in dryness enters the chamber (36), the drying efficiency is improved.

In the present invention, when the solenoid valve (45) is closed again and the solenoid valve (38) is opened to vacuum by the vacuum pump (44) and dry the chamber (36) while performing the drying process, the moisture remaining on the medical appliances evaporates completely. Therefore, because the moisture existing on the medical appliances being sterilized in the chamber (36) as the above is completely dried, bacteria are unable to propagate.

What is claimed is:

1. A sterilizer for medical appliances which comprises a water reservoir (30) which stores distilled water for sterilization, a chamber (36) for heating the distilled water fed from said water reservoir (30) for sterilization of medical appliances to produce vapor, a cooler (40) for reducing the high temperature of hot vapor produced in said chamber (36), and a vacuum pump (44) for reducing the pressure and moisture in said chamber (36), characterized in that said sterilizer for medical appliances further comprising:
a vapor-liquid separator (42) for separating liquid from wet vapor produced in said chamber (36); and
a muffler (46) for reducing noises generated at the discharge side of said vacuum pump (44).

2. The sterilizer for medical appliances of claim 1, wherein said vapor-liquid separator (42) is arranged between said vacuum pump and said cooler.

3. A method of sterilizing medical appliances by heating distilled water stored in a water reservoir (30) to produce vapor, and discharging the produced vapor by use of vacuum pump (44) to sterilize medical appliances received m a chamber (36), characterized in that moisture is separated from the wet vapor produced in said chamber (36), and the noise generated at said vacuum pump (44) is real

4. The method of sterilizing medical appliances of claim 3, wherein said moisture is separated by decreasing the flow velocity of said wet vapor and colliding said wet vapor with a shield plate.

5. The method of sterilizing medical appliances of claim 3, wherein said noise is reduced by expanding the volume of a tube passage through which noise passes.
